(19) United States
(12) Patent Application Publication
(10) Pub. No.: US 2006/0177494 A1
(43) Pub. Date: Aug. 10, 2006

(54) Coated microprojections having reduced variability and method for producing same

(76) Inventors: Micheal Cormier, Mountain View, CA (US); Mahmoud Ameri, Fremont, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/341,832

(22) Filed: Jan. 27, 2006

Related U.S. Application Data
(60) Provisional application No. 60/649,888, filed on Jan. 31, 2005.

(51) Int. Cl. A61K 9/70; A61M 31/00

(52) U.S. Cl. 424/449; 604/500

(57) ABSTRACT

The present invention provides methods and devices for reducing the coating variability of a transdermal microprojection delivery device. The device has one or more stratum corneum-piercing microprojections, wherein each microprojection has a maximum width located in the range of approximately 25% to 75% of the length of the microprojection and wherein the microprojection has a minimum width proximal to the maximum width. Preferably, the microprojection has a coating of a biologically active agent that at a minimum extends to at least approximately 75% of the distance from the distal tip to a location corresponding to the maximum width and at most extends up to approximately 90% of the length of the microprojection.

### CROSS REFERENCE TO RELATED APPLICATIONS

This application claims the benefit of U.S. Provisional Application Ser. No. 60/649,888, filed Jan. 31, 2005.

### FIELD OF THE PRESENT INVENTION

The present invention relates to devices and methods for transdermally delivering a biologically active agent using a coated microprojection array. More particularly, the invention relates to devices and methods for reducing the variability in the amount of active agent coated on the microprojections, thus improving the consistency of delivered amount.

### BACKGROUND OF THE INVENTION

Active agents (or drugs) are most conventionally administered either orally or by injection. Unfortunately, many active agents are completely ineffective or have radically reduced efficacy when orally administered, since they either are not absorbed or are adversely affected before entering the bloodstream and thus do not possess the desired activity. On the other hand, the direct injection of the agent into the bloodstream, while assuring no modification of the agent during administration, is a difficult, inconvenient, painful and uncomfortable procedure which sometimes results in poor patient compliance.

As an alternative, transdermal delivery provides for a method of administering biologically active agents that would otherwise need to be delivered via hypodermic injection, intravenous infusion or orally. Transdermal delivery, when compared to oral delivery, avoids the harsh environment of the digestive tract, bypasses gastrointestinal drug metabolism, reduces first-pass effects, and avoids the possible deactivation by digestive and liver enzymes.

The word “transdermal,” as used herein, is a generic term that refers to the delivery of an active agent (e.g., a nucleic acid or other therapeutic agent such as a drug) through the skin to the local tissue or systemic circulatory system without substantial cutting or piercing of the skin, such as cutting with a surgical knife or piercing the skin with a hypodermic needle.

Transdermal agent delivery includes delivery via passive diffusion as well as by external energy sources, including electricity (e.g., iontophoresis) and ultrasound (e.g., phonophoresis). While most agents will diffuse across both the stratum corneum and the epidermis, the rate of diffusion through the stratum corneum is often the limiting step. Many compounds, in order to achieve a therapeutic dose, require higher delivery rates than can be achieved by simple passive transdermal diffusion.

One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal agent flux has been limited, particularly for larger molecules.

There also have been many techniques and systems developed to mechanically penetrate or disrupt the outermost skin layers thereby creating pathways into the skin in order to enhance the amount of agent being transdermally delivered. Illustrative are skin scarification devices, or scarifiers, which typically provide a plurality of tines or needles that are applied to the skin to scratch or make small cuts in the area of application. The agent, such as a vaccine, is applied either topically on the skin, such as disclosed in U.S. Pat. No. 5,487,726, or as a wetted liquid applied to the scarifier tines, such as disclosed in U.S. Pat. Nos. 4,453,926, 4,109,655, and 3,136,314.

Other devices that use tiny skin piercing elements to enhance transdermal agent delivery are disclosed in European Patent EP 0407063A1, U.S. Pat. No. 5,879,326 issued to Godshall, et al., U.S. Pat. No. 3,814,097 issued to Ganderton, et al., U.S. Pat. No. 5,279,544 issued to Gross, et al., U.S. Pat. No. 5,250,023 issued to Lee, et al., U.S. Pat. No. 3,964,482 issued to Gerstel, et al., Reissue 25,637 issued to Kravitz, et al., and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated by reference in their entirety. The piercing elements disclosed in these references generally extend perpendicularly from a thin, flat member, such as a pad or sheet. The piercing elements are typically extremely small, some having dimensions (i.e., a microblade length and width) of only about 25-400 μm and a microblade thickness of only about 5-50 μm. These tiny piercing/cutting elements make correspondingly small microslits/microcuts in the stratum corneum to enhance transdermal agent delivery.

The disclosed systems generally include a reservoir for holding the active agent and a delivery system to transfer the active agent from the reservoir through the stratum corneum, such as by hollow tines or needles.

Alternatively, a formulation containing the active agent can be coated on the microprojections. Illustrative are the systems disclosed in U.S. Patent Applications No. 2002/0132054, 2002/0193729, 2002/0177839, 2002/0128599, and 10/045,842, which are fully incorporated by reference herein. Coated microprojection systems eliminate the necessity of a separate physical reservoir and the development of an agent formulation or composition specifically for the reservoir.

However, one challenge associated with the noted method of delivery lies in achieving a reproducible dose of the coated agent. Specifically, conventional means of coating can, and in many instances will, result in a variation in the amount of active agent loaded onto the delivery device. For example, depending upon the coating method employed, there can be substantial variations in the overall surface area of each microprojection that receives the coating. As a result, there is an inherent variability in the amount of active agent that is coated on the microprojection device.

As such, it is an object of this invention to provide methods and compositions for facilitating transdermal delivery of biologically active agents using microprojection devices.

It is a further object of the invention to provide a device that reduces the variability in the amount of active agent coated on the microprojections.

It is another object of the invention to a method of delivering a more consistent amount of a biologically active agent using a coated microprojection device.

It is yet another objection of the invention to provide a device and method that reduces the standard deviation in the average coating depth.

### SUMMARY OF THE INVENTION

In accordance with the above objects and those that will be mentioned and will become apparent below, one aspect of the invention comprises a transdermal delivery device comprising a microprojection member having at least one stratum corneum-piercing microprojection, wherein the microprojection has a length extending from a distal tip to a proximal end, wherein the microprojection has a maximum width located at a position in the range of approximately 25% to 75% of the length of the microprojection from the distal tip, and wherein the microprojection has a minimum width proximal to the maximum width.

In some embodiments of the invention, the microprojection has a minimum width in the range of approximately 20% to 80% of the maximum width, and more preferably, in the range of approximately 30% to 70% of the maximum width. In one embodiment, the minimum width is approximately 50% of the maximum width. In another embodiment, the microprojection has a horizontal cross-sectional area proximate the minimum width that is in the range of approximately 30% to 70% of the horizontal cross-sectional area at the maximum width.

In some embodiments, the microprojection has a substantially constant horizontal cross-sectional area from the minimum width to the proximal end. Alternatively, the microprojection has an increasing horizontal cross-sectional area from the minimum width to the proximal end.

In yet another embodiment of the invention, the microprojection has a hexagonally shaped horizontal cross section. Additionally, the microprojection can have a tapered thickness at the distal end.

Preferably, the delivery devices of the invention further comprise a coating of a biologically active agent applied to the microprojection from the distal tip to at least approximately 75% of the distance from the distal tip to a location corresponding to the maximum width. In such embodiments, the coating can be applied to up to approximately 90% of the length of the microprojection, measured from the distal tip. In one embodiment of the invention, the coating comprises a formulation having a biologically active agent selected from the group consisting of ACTH, amylin, angiotensin, angiogenin, anti-inflammatory peptides, BNP, calcitonin, endorphins, endothelin, GLIP, Growth Hormone Releasing Factor (GRF), hirudin, insulin, insulinotropin, neuropeptide Y, PTH, VIP, growth hormone release hormone (GHRH), octreotide, pituitary hormones (e.g., hGH), ANF, growth factors, such as growth factor releasing factor (GFRF), bMSH, somatostatin, platelet-derived growth factor releasing factor, human chorionic gonadotropin, erythropoietin, glucagon, hirulog, interferon alpha, interferon beta, interferon gamma, interleukins, granulocyte macrophage colony stimulating factor (GM-CSF), granulocyte colony stimulating factor (G-CSF), menotropins (urofollitropin (FSH) and LH)), streptokinase, tissue plasminogen activator, urokinase, ANF, ANP, ANP clearance inhibitors, antidiuretic hormone agonists, calcitonin gene related peptide (CGRP), IGF-1, pentigetide, protein C, protein S, thymosin alpha-1, vasopressin antagonists analogs, alpha-MSH, VEGF, PYY, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and analogs and derivatives derived from the foregoing and mixtures thereof.

In another embodiment of the invention, the biologically active agent comprises a formulation having an immunologically active agent selected from the group consisting of proteins, polysaccharide conjugates, oligosaccharides, lipoproteins, subunit vaccines, Bordetella pertussis (purified, recombinant), Clostridium tetani (purified, recombinant), Corynebacterium diphtheriae (purified, recombinant), recombinant DPT vaccine, Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F conjugated to meningococcal B OMP, glycoconjugate [4, 6B, 9V, 14, 18C, 19F, 23F conjugated to CRM197, glycoconjugate [1, 4, 5, 6B, 9V, 14, 18C, 19F, 23F conjugated to CRM1970, Treponema pallidum (surface lipoproteins), Varicella zoster virus (subunit, glycoproteins), Vibrio cholerae (conjugate lipopolysaccharide), whole virus, bacteria, weakened or killed viruses, cytomegalo virus, hepatitis B virus, hepatitis C virus, human papillomavirus, rubella virus, varicella zoster, weakened or killed bacteria, bordetella pertussis, clostridium tetani, corynebacterium diphtheriae, group A streptococcus, legionella pneumophila, neisseria meningitidis, pseudomonas aeruginosa, streptococcus pneumoniae, treponema pallidum, vibrio cholerae, flu vaccines, lyme disease vaccine, rabies vaccine, measles vaccine, mumps vaccine, chicken pox vaccine, small pox vaccine, hepatitis vaccine, pertussis vaccine, diphtheria vaccine, nucleic acids, single-stranded and double-stranded nucleic acids, supercoiled plasmid DNA, linear plasmid DNA, cosmids, bacterial artificial chromosomes (BACs), yeast artificial chromosomes (YACs), mammalian artificial chromosomes, and RNA molecules.

The invention also comprises methods of applying a coating containing a biologically active agent on a transdermal delivery device, generally including the steps of providing a microprojection member having at least one stratum corneum-piercing microprojection, wherein the microprojection has a length extending from a distal tip to a proximal end, wherein the microprojection has a maximum width located in the range of approximately 25% to 75% of the length of the microprojection measured from the distal tip of the microprojection, and wherein the microprojection has a minimum width proximal to the maximum width; applying a biologically active agent formulation to the microprojection; and drying the formulation to form a coating. Preferably, the step of applying the formulation comprises roller coating.

In one embodiment of the invention, the step of applying the formulation comprises applying the formulation to the microprojection from the distal tip to at least approximately 75% of the distance from the distal tip to a location corresponding to the maximum width. Additionally, the step of applying the formulation comprises applying the formulation to up to approximately 90% of the length of the microprojection, measured from the distal tip.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a microprojection member having a coating deposited on the microprojections, according to the invention;

FIG. 2 is a front view of a microprojection, according to the invention;

FIG. 3 is a side view of a microprojection member, according to the invention;

FIG. 4 is a cross-sectional view of the microprojection shown in FIGS. 2 and 3, taken at line 4A-4A, according to the invention;

FIG. 5 is a schematic illustration of a microprojection having reduced horizontal cross-sectional area proximal to the maximum width, according to the invention;

FIG. 6 is a cross-sectional view of the microprojection shown in FIG. 5, taken at line 6A-6A;

FIGS. 7 and 8 are schematic illustrations of microprojection designs for comparison to the designs of the invention;

FIG. 9 is a graphical illustration of microprojection horizontal cross-sectional area as a function of the distance from the distal tip of the microprojection for the microprojection designs shown in FIGS. 2, 7 and 8;

FIG. 10 is a graphical illustration of microprojection coated area as a function of the coating depth for the designs shown in FIGS. 2, 7 and 8;

FIG. 11 is a graphical illustration of a statistical distribution of predicted average coating depth on a microprojection;

FIG. 12 is a graphical illustration of the predicted standard deviation of coated area as a function of coating depth for the microprojection designs shown in FIGS. 2, 7 and 8;

FIG. 13 is a graphical illustration of the predicted standard deviation of coated area as a function of coating depth for the microprojection designs shown in FIGS. 2 and 5, according to the invention;

FIG. 14 is a graphical illustration of microprojection coated area as a function of the coating depth for the microprojection designs shown in FIGS. 2 and 5, according to the invention;

FIG. 15 is a graphical illustration of microprojection coated area as a function of the coating depth at varying tip angles for the microprojection design shown in FIG. 2, according to the invention;

FIGS. 16-28 illustrate microprojection designs for reducing the variability of coating amount, according to the invention;

FIGS. 29-34 illustrate further microprojection designs having a vertical minimum cross-sectional area as shown in FIG. 6, according to the invention;

FIGS. 35 and 36 illustrate further microprojection designs having a horizontal cross-sectional area that increases proximal to the minimum horizontal cross-sectional area, according to the invention; and

FIGS. 37 and 38 illustrate yet additional microprojection designs for reducing the variability of coating amount, according to the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### Definitions

The term “transdermal”, as used herein, means the delivery of an agent into and/or through the skin for local or systemic therapy.

The term “biologically active agent”, as used herein, refers to a composition of matter or mixture containing an active agent or drug, which is pharmacologically effective when administered in a therapeutically effective amount. Preferred active agents are nucleic acids, such as oligonucleotides and polynucleotides. Alternatively, biologically active agents can comprise small molecular weight compounds, polypeptides, proteins and polysaccharides.

It is to be understood that more than one biologically active agent can be incorporated into the agent source and/or coatings of this invention, and that the use of the term “active agent” in no way excludes the use of two or more such active agents or drugs.

As used herein, the term “microprojection array,” “microprojection member,” and the like, all refer to a device for delivering an active agent into or through the skin that comprises a plurality of microprojections on which the active agent can be coated. The term “microprojections” refers to piercing elements that are adapted to pierce or cut through the stratum corneum into the underlying epidermis layer, or epidermis and dermis layers, of the skin of a living animal, particularly a human. Typically the piercing elements have a blade length of less than 1000 μm, and preferably less than 500 μm. The microprojections typically have a width of about 75 μm to 500 μm and a thickness of about 5 μm to 50 μm.

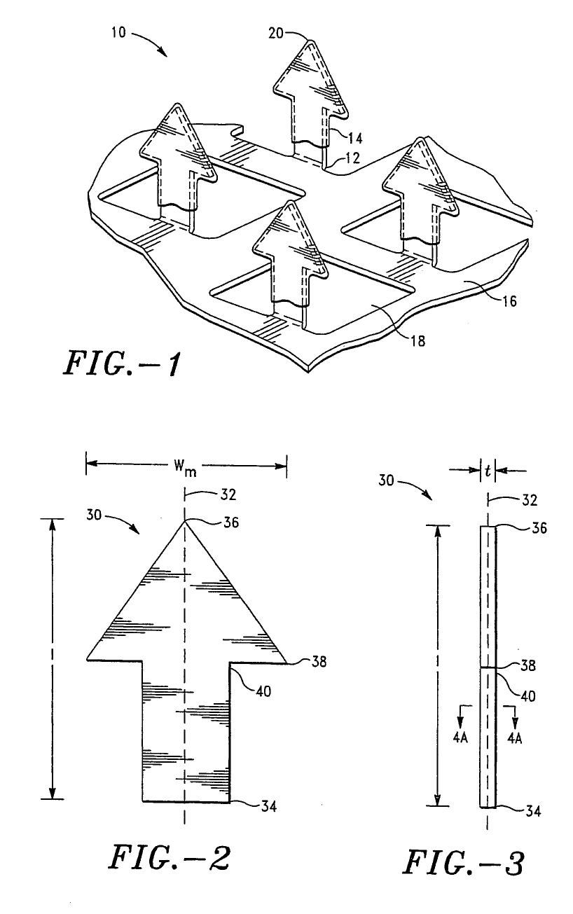

The microprojections can be formed in different shapes, pursuant to the dimensional constraints described below, such as needles, hollow needles, blades, pins, punches, and combinations thereof. The microprojection member can be formed by etching or punching a plurality of microprojections from a thin sheet and folding or bending the microprojections out of the plane of the sheet to form a configuration, such as that shown in FIG. 1. The microprojection member can also be formed in other known manners, such as by forming one or more strips having microprojections along an edge of each of the strip(s).

Exemplary methods of forming metal microprojection are disclosed in Trautman et al., U.S. Pat. No. 6,083,196; Zuck, U.S. Pat. No. 6,050,988; and Daddona et al., U.S. Pat. No. 6,091,975; the disclosures of which are incorporated by reference herein in their entirety.

Other microprojection members that can be used with the present invention are formed by etching silicon using silicon chip etching techniques or by molding plastic using etched micro-molds. Silicon and plastic microprojection members are disclosed in Godshall et al., U.S. Pat. No. 5,879,326; the disclosure of which is incorporated by reference herein.

Presently preferred characteristics of the microprojection members of the invention include a microprojection density in the range of approximately 10 to 2000 per cm², a microprojection length in the range of approximately 50 to 500 μm, a microprojection maximum width in the range of approximately 20 to 300 μm, and a microprojection thickness in the range of approximately 10 to 50 μm.

As used herein, the terms “deliver,” “delivering,” and all variations thereof, refer to and include any means by which an active agent can be administered into or through the skin.

As used herein, the term “thickness,” as it relates to coatings, refers to the average thickness of a coating as measured over substantially all of the portion of a substrate that is covered with the coating.

Referring to FIG. 1, there is shown one embodiment of stratum corneum-piercing microprojection member 10 for use with the present invention. As shown in FIG. 1, member 10 includes a plurality of microprojections 12 having a coating 14 disposed thereon. The coating 14 is preferably applied after the microprojections 12 are formed. Microprojections 12 extend at substantially a 90° angle from a substrate, such as sheet 16, having openings 18. Microprojections 12 are preferably formed by etching or punching a plurality of microprojections 12 from a thin metal sheet 16 and bending the microprojections 12 out of a plane of the sheet. Metals such as stainless steel, titanium and nickel titanium alloys are preferred.

According to the invention, the coating 14 preferably covers the microprojection from the distal tip 20 for an amount in the range of approximately 75% of the distance from the distal tip to a location corresponding to the maximum width and up to 90% of the overall length. Specific minimum coating depths are discussed below. Preferably, the entire length of the microprojection is not covered. Due to the inherent variability in coating depth, attempts to cover the entire microprojection risks contamination of the substrate with the active agent. In turn, this will lead to irreproducible loading and delivery amounts.

According to the invention, the coating 14 can be applied to the microprojections 12 by a variety of known methods. Preferably, the coating is only applied to those portions the microprojection member 10 or microprojections 12 that pierce the skin (e.g., tips).

A presently preferred means of coating the microprojections of the invention is roller coating as disclosed in U.S. application Ser. No. 10/099,604 (Pub. No. 2002/0132054), which is incorporated by reference herein in its entirety. As discussed in detail in the noted application, the disclosed roller coating method provides a smooth coating that is not easily dislodged from the microprojections 12 during skin piercing.

An alternative coating means is dip-coating. Dip-coating can be described as a means to coat the microprojections by partially or totally immersing the microprojections 12 into a coating solution. By use of a partial immersion technique, it is possible to limit the coating 14 to only the tips of the microprojections 12.

Yet another means of coating the microprojections is “dry-coating.” This refers to any process by which a solution that contains one or more agents of interest is applied to a surface of a solid substrate and by which substantially all of the liquid is then removed from the solution of the one or more agents of interest. The terms “dry-coated” and “dry-coat,” and all variations thereof refer to the resultant solid coating produced by the dry coating process.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 12. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

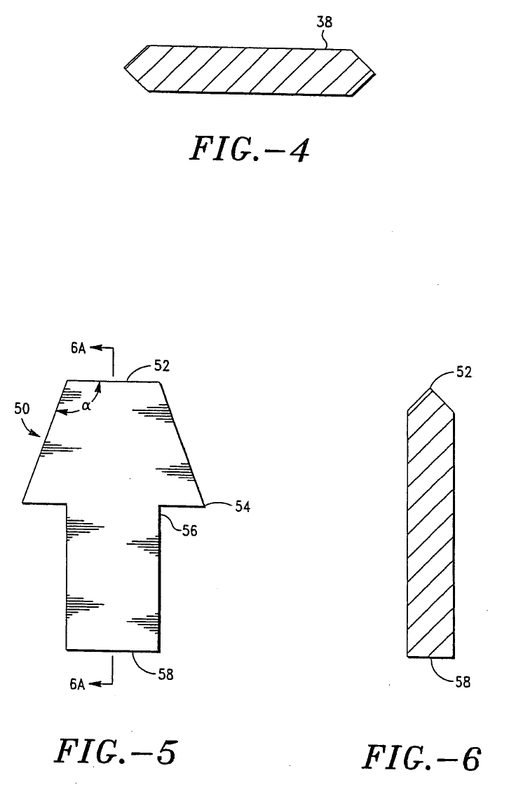

A presently preferred microprojection design of the invention is shown in FIGS. 2 and 3, in which the microprojection 30 has standard dimensions including a major axis 32 extending the length of the microprojection 30 from the proximal end 34 that is secured to the substrate of the microprojection member to the distal end 36 at the tip of the microprojection 30. The term “horizontal cross-sectional area” refers to the area of the cross section of a microprojection perpendicular to the major axis 32. The “horizontal maximum cross-sectional area,” shown in FIGS. 2 and 3, is taken at 4A-4A and shown in FIG. 4.

The term “microprojection maximum width,” wₘ, refers to the maximum dimension perpendicular to axis 32 of microprojection 30 and is shown at location 38. According to the invention, the microprojection maximum width corresponds to the location of the horizontal maximum cross-sectional area. Conversely, the term “microprojection minimum width” does not refer to the tip of the microprojection, but rather the minimum dimension that is coplanar with the maximum width and is perpendicular to axis 32 of the microprojection 30 in a region between location 38 and proximal end 34. The minimum width can also be located at the location of the proximal end 34 of the microprojection.

As shown in FIGS. 2 and 3, microprojection 30 preferably has a constant minimum width extending from location 40 to proximal end 34. Microprojection 30 also has an overall length, l, along axis 32. Finally, the term “microprojection thickness,” t, refers to the dimension perpendicular to both the axis 32 and the width of the microprojection 30. For example, the microprojection thickness can be the thickness of the metallic foil when the microprojections are obtained by etching and forming technology.

As stated above, the present invention is directed to microprojection designs and methods having reduced coating variability. To achieve minimal coating variability, the horizontal cross-sectional area preferably increases from the distal tip 36 to location 38 of maximum width. More preferably, location 38 of maximum width is located in the range of approximately 25% to 75% of the length of the microprojection, as measured from distal tip 36.

Preferably, the horizontal cross-sectional area of microprojection 30 decreases proximally from location 38, the maximum width, to location 40, a minimum width. As shown in this embodiment, the horizontal cross-sectional area remains substantially constant from the minimum width location 40 to the proximal end 34. Alternatively, as described below with reference to FIGS. 34 and 35, the horizontal cross-sectional area can increase again in the region proximal to the minimum width.

The minimum width at location 40 of microprojection 30 is preferably in the range of approximately 20% to 80% of the maximum width, and more preferably, in the range of approximately 30% to 70% of the maximum width. In one embodiment, the minimum width at location 40 is approximately 50% of the maximum width at location 38. Alternatively, the horizontal cross-sectional area at the minimum width location 40 is in the range of approximately 30% to 70% of the horizontal cross-sectional area at the maximum width location 38.

The microprojections of the invention are preferably obtained by etching the microprojection from a thin metallic sheet and forming them perpendicular to the metallic sheet. The horizontal cross-sectional area of the microprojection preferably comprises a square, a rectangle, or a polygon. For example, in the embodiment illustrated in FIG. 4, the cross section taken from microprojection 30 at line 4A-4A shows a hexagonal cross section. Alternatively, the horizontal cross-sectional area can comprise a circle, an ellipse or an ellipsoid. Preferably, the horizontal cross section shape maximizes the area of the microprojection for subsequent coating and skin penetration. One having ordinary skill in the art will recognize that such conformations can readily be obtained during the etching process.

FIG. 5 shows a microprojection 50 embodying features of the invention, whereby the horizontal cross-sectional area increases from the distal tip 52 to a maximum width at location 54, located in the range of approximately 25% to 75% of the length of the microprojection 50, as measured from distal tip 52. Proximal to the maximum width, there is a minimum width location 56. As shown in this embodiment, the minimum width extends from location 56 to proximal end 58.

Microprojection 50 differs from microprojection 30 in that it presents a linear tip 52 forming two angles, rather than a point. To obtain satisfactory stratum corneum-piercing characteristics, the thickness of tip 52 should preferably taper as shown in FIG. 6, which corresponds to the cross section of microprojection 50 taken at line 6A-6A. Such a taper can be achieved by any suitable means, including a method of double etching a metallic sheet.

Preferably, tip 52 has a dimension in the range of approximately 5 to 100 μm, more preferably, in the range of 20 to 80 μm. Also preferably, the two angles a, formed by linear tip 52 are in the range of approximately 100° to 145°. In one embodiment, linear tip 52 is 60 μm and forms two 120° angles.

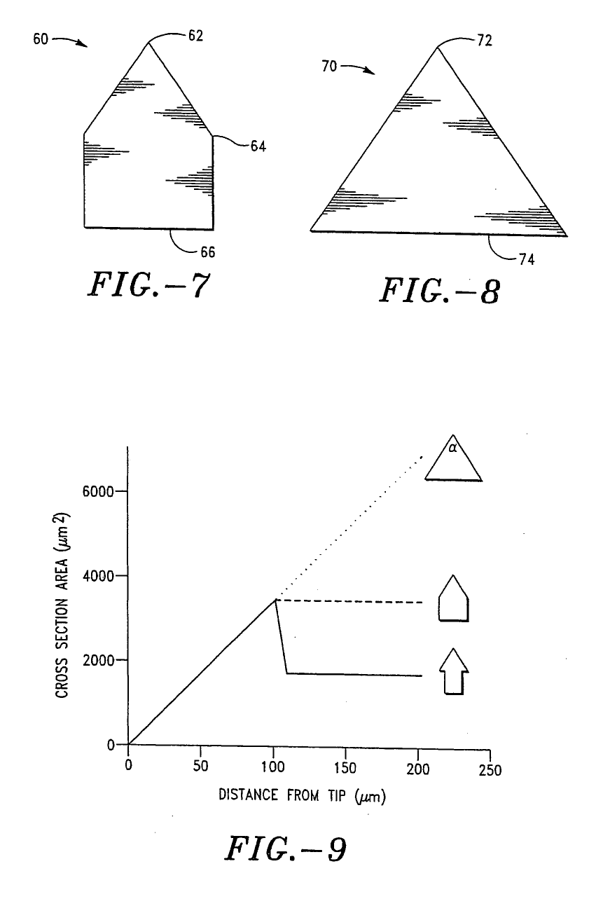

FIGS. 7 and 8 show microprojection designs for comparison to demonstrate the reduction in coating variability effected by the invention. As illustrated in FIG. 7, microprojection 60 has a horizontal cross-sectional area that increases from the distal tip 62 to a maximum width location 64, which is located in the range of approximately 25% to 75% of the length of the microprojection 60. In this design, however, there is no minimum horizontal cross-sectional area as the horizontal cross-sectional area remains constant from the maximum width location 64 to the proximal end 66 of microprojection 60. Referring now to FIG. 8, there is shown another microprojection design wherein the microprojection 70 has a horizontal cross section that increases constantly from the distal tip 72 to the proximal end 74.

For the three different microprojection designs shown in FIGS. 2, 7 and 8, the horizontal cross-sectional area can be calculated as a function of the distance from the tip of the microprojection. These results are shown in FIG. 9. These calculations were derived based upon a microprojection length of 200 μm, a tip angle of 60°, a rectangular cross-sectional area, and a microprojection thickness of 30 μm. For the designs shown in FIGS. 2 and 7, the horizontal maximum cross-sectional area is located at 100 μm, or 50% of the length of the microprojection as measured from the distal tip. For the design shown in FIG. 7, the horizontal maximum cross-sectional area is located at 200 μm, which corresponds to 100% of the length of the microprojection or the proximal end 66 and the tip 62 has an angle of 60°. For the designs shown in FIGS. 2 and 7, the maximum width was 115 μm. For the design shown in FIG. 2, the minimum width of microprojection 30 is 58 μm, of approximately 50% of the maximum width.

For each of the noted configurations, there is a region of increasing horizontal maximum cross-sectional area. However, only FIG. 2 shows a microprojection design embodying features of the invention by having a minimum width at location 40 proximal to the maximum width location 38.

Further, the surface area of the microprojections can be calculated as a function of the distance from the tip of the microprojection, as shown in FIG. 10. The amount of active agent coated onto the microprojection is roughly proportional to the surface area being coated during the coating process.

As discussed above, there is an inherent variability of the amount of coating deposited on the microprojection during coating. This variability is related to differences in coating distance from the tip of the microprojection, or coating depth. FIG. 11 shows the Gaussian distribution predicted for an average coating depth of 80 μm, with a standard deviation of approximately 12 μm.

FIG. 12 illustrates the predicted standard deviation, which is expressed as the percentage of the average coated area, for various average coating depths associated with the designs shown in FIGS. 2, 7 and 8. The noted results demonstrate that the variability of the coated area decreases from the tip of the microprojection as a function of the coating depth. Moreover, microprojection 30 (shown in FIG. 2) exhibits a dramatic decrease in the standard deviation of the coating depth compared with the designs shown in FIGS. 7 and 8. This decrease starts for coating depths that are at least approximately 75% of the distance from distal tip 36 and the location 38 corresponding to the maximum width.

The results discussed above are based upon an assumed standard deviation of 12 μm, which corresponds to extremes of approximately ±20 μm. One having skill in the art that this variability will depend upon the precision of the coating apparatus. However, the microprojection designs will reduce the coating variability, making the invention applicable so long as there is any variability in the coating method.

FIG. 13 shows a further reduction in the predicted standard deviation of the coated area can be achieved with the microprojection design shown in FIG. 5, with respect to the microprojection design shown in FIG. 2. The reduction is achieved by increasing the amount of surface area distal to location corresponding to the minimum width of the microprojection. This increase in the coated surface area is shown in FIG. 14.

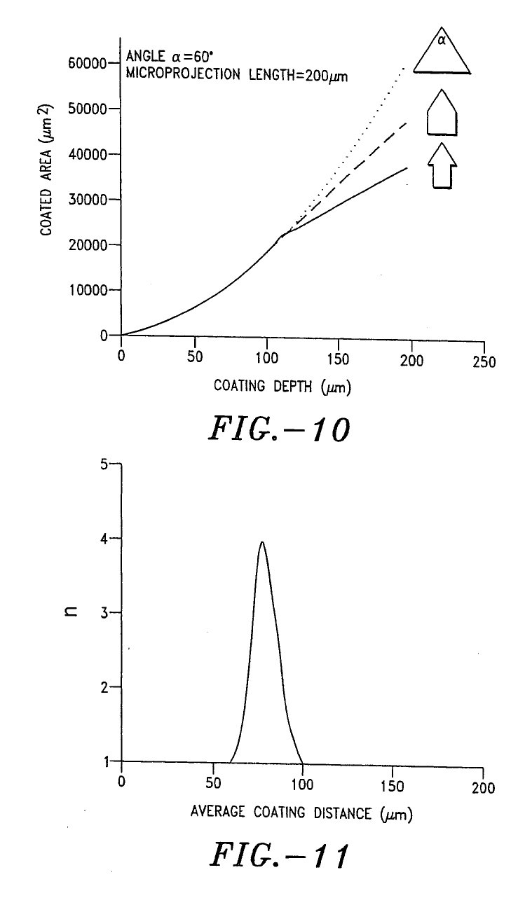

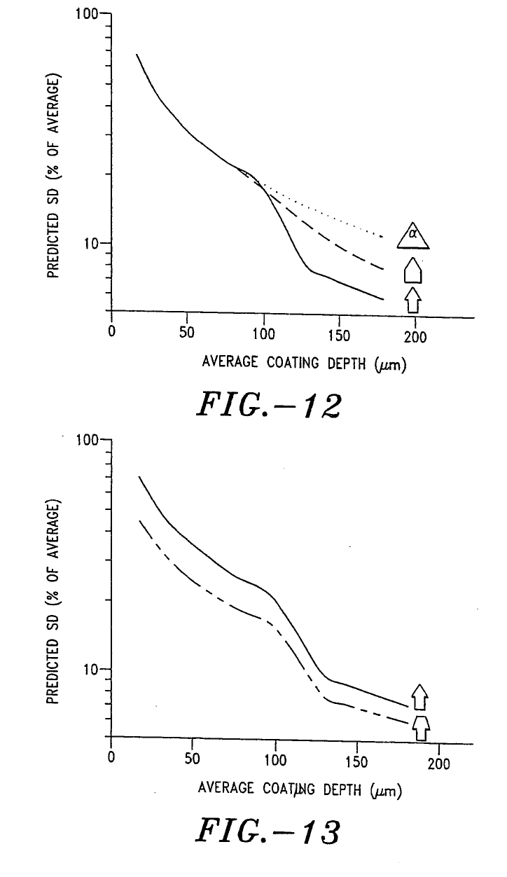

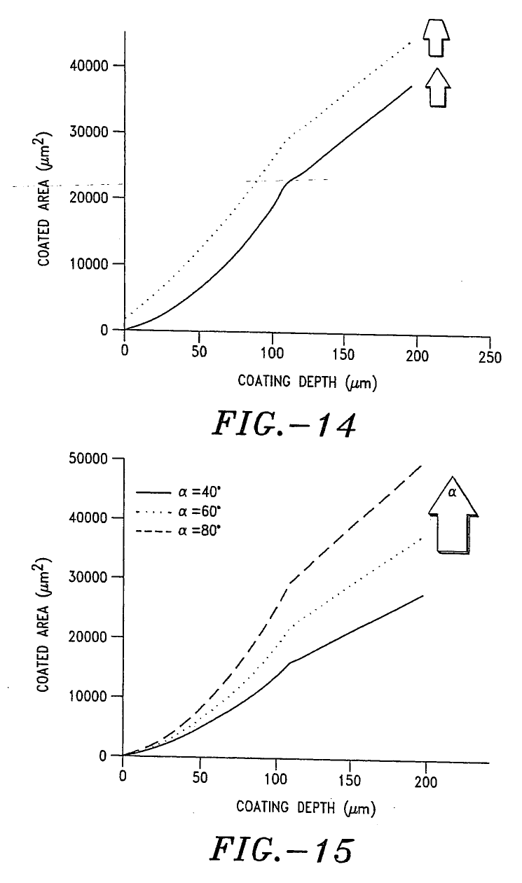

Alternatively, the coated area of a microprojection having the general configuration in FIG. 4 can be increased by increasing the tip angle. As shown in FIG. 15, increasing the tip angle causes a corresponding increase in coated area. However, standard deviation was not affected by the changing tip angle.

From the above examples, coating variability is reduced by employing microprojection designs wherein the horizontal cross-sectional area increases from the tip of the microprojection to the maximum width at a location in the range of approximately 25% to 75% of the length of the microprojection. Below 25%, the area available for coating is generally inadequate. A design having a maximum width located more that 75% of the distance from the tip would require applying too deep a coating, significantly increasing the risk of applying coating to the sheet. Proximal to the maximum width, the cross-sectional area of the microprojection should decrease to a location corresponding to the minimum width. From the minimum width to the proximal end, the microprojection can maintain the minimum width or can increase. Alternatively, the minimum width is located at the location of the proximal end of the microprojection.

The microprojection designs of the invention are preferably coated with a formulation that forms a solid coating when applied to the surface of the microprojection. The coatings, at a minimum, cover at least approximately 75% of the distance between the tip of the microprojection and the maximum width and at a maximum cover up to approximately 90% of the total length of the microprojection, measured from the distal tip. Applying a coating to less than approximately 75% of the distance between the tip and maximum width does not significantly reduce the standard deviation of average coating depth. Applying a coating to more than approximately 90% of the total length of the microprojection presents an undesirable risk of contaminating the substrate from which the microprojection extends, resulting in increased variability.

Additional microprojection designs that exhibit maximum and minimum widths are shown in FIGS. 16-28. The microprojections 80a-80M have a distal tip 82 and a horizontal cross-sectional area that increases to a horizontal maximum cross-sectional area at maximum width location 84. The microprojections 80a-80M also have a minimum width location 86, in between maximum width location 84 and proximal end 88. These designs embody features of the invention and correspondingly provide reduced coating variability.

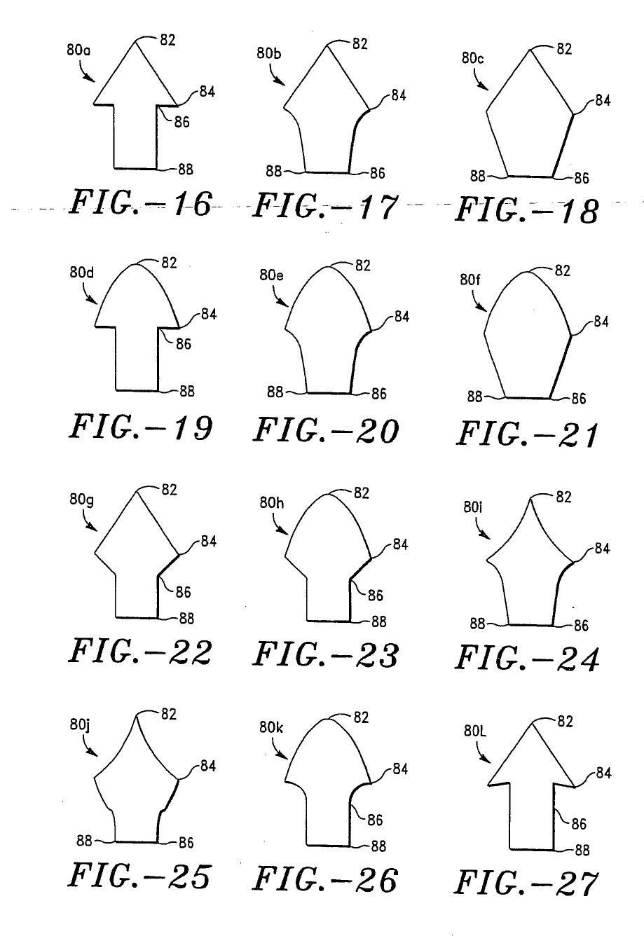

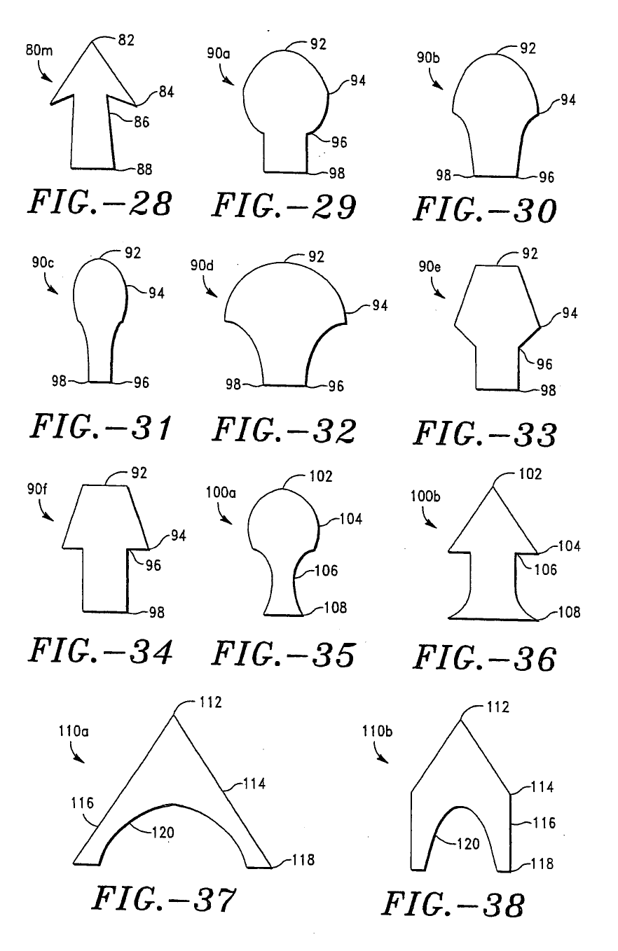

Further microprojection designs are shown in FIGS. 29-34. The shown microprojections 90a-90f have a distal tip 92 and a horizontal cross-sectional area that increases to a horizontal maximum cross-sectional area at maximum width location 94. The microprojections 90a-90f also have a minimum width location 96, in between maximum width location 94 and proximal end 98. Due to the generally broader distal tips 82, the noted design configurations preferably have a tapered thickness distal end, such as shown in FIG. 6.

The microprojection designs shown in FIGS. 35 and 36 also embody features of the invention. As shown, the microprojections 100a and 100b have a distal tip 102 and a horizontal cross-sectional area that increases to a horizontal maximum cross-sectional area at maximum width location 104. The microprojections 100a and 100b also have a minimum width location 106, in between maximum width location 104 and proximal end 108. Proximal to minimum width location 106, the horizontal cross-sectional area increases again.

Finally, FIGS. 37 and 38 show yet other suitable microprojection configurations embodying features of the invention. In these embodiments, the microprojections 110a and 110b have a distal tip 112 and a horizontal cross-sectional area that increases to a horizontal maximum cross-sectional area at maximum width location 114. The microprojections 110a and 110b also have a minimum width location 116, in between maximum width location 114 and proximal end 118. The minimum width location 116 is formed by void 120 adjacent proximal end 118. Void 120 creates a maximum width location 114 distal to void 120, with a corresponding horizontal maximum cross-sectional area.

In one aspect of the invention, the biologically active agent comprises a therapeutic agent in all the major therapeutic areas including, but not limited to, anti-infectives, such as antibiotics and antiviral agents; analgesics, including buprenorphine and analgesic combinations; anesthetics; anorexics; antiarthritics; antiasthmatic agents, such as terbutaline; anticonvulsants; antidepressants; antidiabetic agents; antidiarrheals; antihistamines; anti-inflammatory agents; antimigraine preparations; antimotion sickness preparations, such as scopolamine and ondansetron; antinauseants; antineoplastics; antiparkinsonism drugs; antipruritics; antipsychotics; antipyretics; antispasmodics, including gastrointestinal and urinary; anticholinergics; sympathomimetrics; xanthine derivatives; cardiovascular preparations, including calcium channel blockers such as nifedipine; beta blockers; beta-agonists, such as dobutamine and ritodrine; antiarrythmics; antihypertensives, such as atenolol; ACE inhibitors, such as ranitidine; diuretics; vasodilators, including general, coronary, peripheral, and cerebral; central nervous system stimulants; cough and cold preparations; decongestants; diagnostics; hormones, such as parathyroid hormone; hypnotics; immunosuppressants; muscle relaxants; parasympatholytics; parasympathomimetrics; prostaglandins; proteins; peptides; psychostimulants; sedatives; and tranquilizers. Other suitable agents include vasoconstrictors, anti-healing agents and pathway patency modulators. One or more biologically active agents can also be combined as desired.

In a preferred embodiment, the biologically active agent is selected from the group consisting of ACTH, amylin, angiotensin, angiogenin, anti-inflammatory peptides, BNP, calcitonin, endorphins, endothelin, GLIP, Growth Hormone Releasing Factor (GRF), hirudin, insulin, insulinotropin, neuropeptide Y, PTH , VIP, growth hormone release hormone (GHRH), octreotide, pituitary hormones (e.g., hGH), ANF, growth factors, such as growth factor releasing factor (GFRF), bMSH, somatostatin, platelet-derived growth factor releasing factor, human chorionic gonadotropin, erythropoietin, glucagon, hirulog, interferon alpha, interferon beta, interferon gamma, interleukins, granulocyte macrophage colony stimulating factor (GM-CSF), granulocyte colony stimulating factor (G-CSF), menotropins (urofollitropin (FSH) and LH)), streptokinase, tissue plasminogen activator, urokinase, ANF, ANP, ANP clearance inhibitors, antidiuretic hormone agonists, calcitonin gene related peptide (CGRP), IGF-1, pentigetide, protein C, protein S, thymosin alpha-1, vasopressin antagonists analogs, alpha-MSH, VEGF, PYY, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and analogs and derivatives derived from the foregoing and mixtures thereof.

Other suitable biologically active agents include immunologically active agents, such as vaccines and antigens in the form of proteins, polysaccharide conjugates, oligosaccharides, and lipoproteins. Specific subunit vaccines in include, without limitation, Bordetella pertussis (purified, recombinant), Clostridium tetani (purified, recombinant), Corynebacterium diphtheriae (purified, recombinant), recombinant DPT vaccine, Cytomegalovirus (glycoprotein subunit), Group A streptococcus (glycoprotein subunit, glycoconjugate Group A polysaccharide with tetanus toxoid, M protein/peptides linke to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F conjugated to meningococcal B OMP, glycoconjugate [4, 6B, 9V, 14, 18C, 19F, 23F conjugated to CRM197, glycoconjugate [1, 4, 5, 6B, 9V, 14, 18C, 19F, 23F conjugated to CRM1970, Treponema pallidum (surface lipoproteins), Varicella zoster virus (subunit, glycoproteins), and Vibrio cholerae (conjugate lipopolysaccharide).

Suitable immunologically active agents also include nucleic acids, such as single-stranded and double-stranded nucleic acids, supercoiled plasmid DNA, linear plasmid DNA, cosmids, bacterial artificial chromosomes (BACs), yeast artificial chromosomes (YACs), mammalian artificial chromosomes, and RNA molecules.

For storage and application (in accordance with one embodiment of the invention), the microprojection member 10 is preferably suspended in a retainer ring by adhesive tabs, as described in detail in Co-Pending U.S. application Ser. No. 09/976,762 (Pub. No. 2002/0091357), which is incorporated by reference herein in its entirety.

After placement of the microprojection member 10 in the retainer ring, the microprojection member 10 is applied to the patient's skin. Preferably, the microprojection member 10 is applied to the skin using an impact applicator, such as disclosed in Co-Pending U.S. application Ser. No. 09/976,798, which is incorporated by reference herein in its entirety.

From the foregoing description, one of ordinary skill in the art can easily ascertain that the present invention, among other things, provides an effective and efficient means for enhancing the transdermal flux of a biologically active agent into and through the stratum corneum of a patient.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A transdermal delivery device comprising a microprojection member having at least one stratum corneum-piercing microprojection, wherein said microprojection has a length extending from a distal tip to a proximal end, wherein said microprojection has a maximum width located in the range of approximately 25% to 75% of the length of said microprojection measured from said distal tip of said microprojection, and wherein said microprojection has a minimum width proximal to said maximum width.

2. The device of claim 1, wherein said minimum width is in the range of approximately 20% to 80% of said maximum width.

3. The device of claim 2, wherein said minimum width is in the range of approximately 30% to 70% of said maximum width.

4. The device of claim 3, wherein said minimum width is approximately 50% of said maximum width.

5. The device of claim 1, wherein a horizontal cross-sectional area at said minimum width is in the range of approximately 30% to 70% of a horizontal cross-sectional area at said maximum width.

6. The device of claim 1, wherein said microprojection has a substantially constant horizontal cross-sectional area from a location corresponding to said minimum width to said proximal end.

7. The device of claim 1, wherein said microprojection has increasing horizontal cross-sectional area from a location corresponding to said minimum width to said proximal end.

8. The device of claim 1, further comprising a coating of a biologically active agent applied to said microprojection from said distal tip to at least approximately 75% of the distance from said distal tip to a location corresponding to said maximum width.

9. The device of claim 8, wherein said coating is applied to up to approximately 90% of said length of said microprojection, measured from said distal tip.

10. The device of claim 8, wherein said biologically active agent is selected from the group consisting of ACTH, amylin, angiotensin, angiogenin, anti-inflammatory peptides, BNP, calcitonin, endorphins, endothelin, GLIP, Growth Hormone Releasing Factor (GRF), hirudin, insulin, insulinotropin, neuropeptide Y, PTH, VIP, growth hormone release hormone (GHRH), octreotide, pituitary hormones (e.g., hGH), ANF, growth factors, such as growth factor releasing factor (GFRF), bMSH, somatostatin, platelet-derived growth factor releasing factor, human chorionic gonadotropin, erythropoietin, glucagon, hirulog, interferon alpha, interferon beta, interferon gamma, interleukins, granulocyte macrophage colony stimulating factor (GM-CSF), granulocyte colony stimulating factor (G-CSF), menotropins (urofollitropin (FSH) and LH)), streptokinase, tissue plasminogen activator, urokinase, ANF, ANP, ANP clearance inhibitors, antidiuretic hormone agonists, calcitonin gene related peptide (CGRP), IGF-1, pentigetide, protein C, protein S, thymosin alpha-1, vasopressin antagonists analogs, alpha-MSH, VEGF, PYY, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and analogs and derivatives derived from the foregoing and mixtures thereof.

11. The device of claim 8, wherein said biologically active agent comprises an immunologically active agent selected from the group consisting of proteins, polysaccharide conjugates, oligosaccharides, lipoproteins, subunit vaccines, Bordetella pertussis (purified, recombinant), Clostridium tetani (purified, recombinant), Corynebacterium diphtheriae (purified, recombinant), recombinant DPT vaccine, Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F conjugated to meningococcal B OMP, glycoconjugate [4, 6B, 9V, 14, 18C, 19F, 23F conjugated to CRM197, glycoconjugate [1, 4, 5, 6B, 9V, 14, 18C, 19F, 23F conjugated to CRM1970, Treponema pallidum (surface lipoproteins), Varicella zoster virus (subunit, glycoproteins), Vibrio cholerae (conjugate lipopolysaccharide), whole virus, bacteria, weakened or killed viruses, cytomegalo virus, hepatitis B virus, hepatitis C virus, human papillomavirus, rubella virus, varicella zoster, weakened or killed bacteria, bordetella pertussis, clostridium tetani, corynebacterium diphtheriae, group A streptococcus, legionella pneumophila, neisseria meningitidis, pseudomonas aeruginosa, streptococcus pneumoniae, treponema pallidum, vibrio cholerae, flu vaccines, lyme disease vaccine, rabies vaccine, measles vaccine, mumps vaccine, chicken pox vaccine, small pox vaccine, hepatitis vaccine, pertussis vaccine, diphtheria vaccine, nucleic acids, single-stranded and double-stranded nucleic acids, supercoiled plasmid DNA, linear plasmid DNA, cosmids, bacterial artificial chromosomes (BACs), yeast artificial chromosomes (YACs), mammalian artificial chromosomes, and RNA molecules.

12. The device of claim 1, wherein said microprojection has a hexagonally shaped horizontal cross section.

13. The device of claim 1, wherein said microprojection has a tapered thickness at said distal end.

14. A method of applying a coating of a biologically active agent to a transdermal delivery device comprising the steps of providing a microprojection member having at least one stratum corneum-piercing microprojection, wherein said microprojection has a length extending from a distal tip to a proximal end, wherein said microprojection has a maximum width located in the range of approximately 25% to 75% of the length of said microprojection measured from said distal tip of said microprojection, and wherein said microprojection has a minimum width proximal to said maximum width; applying a formulation of said biologically active agent to said microprojection; and drying said formulation to form a coating.

15. The method of claim 14, wherein the step of applying said formulation comprises roller coating.

16. The method of claim 14, wherein the step of applying said formulation comprises applying said formulation to said microprojection from said distal tip to at least approximately 75% of the distance from said distal tip to a location corresponding to said maximum width.

17. The method of claim 16, wherein the step of applying said formulation comprises applying said formulation to up to approximately 90% of said length of said microprojection, measured from said distal tip.
